(19) United States
(12) Patent Application Publication
(10) Pub. No.: US 2010/0330002 A1
(43) Pub. Date: Dec. 30, 2010

(54) COMPOSITIONS AND METHODS COMPRISING BASIC AMINO ACID PEPTIDES AND PROTEASES

(75) Inventors: Richard Scott Robinson, Belle Mead, NJ (US); Richard J. Sullivan, Atlantic Highlands, NJ (US)

Correspondence Address: COLGATE-PALMOLIVE COMPANY, 909 RIVER ROAD, PISCATAWAY, NJ, 08855, US

(73) Assignee: Colgate-Palmolive Company, New York, NY (US)

(21) Appl. No.: 12/866,663

(22) PCT Filed: Feb. 6, 2009
(86) PCT No.: PCT/US09/33285

§ 371 Date: Aug. 6, 2010

Related U.S. Application Data
(62) Division of application No. 61/027,431, filed on Feb. 8, 2008.
Application No. 61/027,431 is a division of application No. 61/027,432, filed on Feb. 8, 2008.
Application No. 61/027,432 is a division of application No. 61/027,420, filed on Feb. 8, 2008.
Application No. 61/027,420 is a division of application No. 61/027,435, filed on Feb. 8, 2008.
Application No. 61/027,435 is a division of application No. 61/027,442, filed on Feb. 9, 2008.
Application No. 61/027,442 is a division of application No. 61/027,584, filed on Feb. 11, 2008.

(51) Int. Cl. A61K 8/66; A61K 8/97; A61K 9/68; A61Q 11/00

(52) U.S. Cl. 424/48; 424/50

(57) ABSTRACT

The present invention is directed to compositions comprising peptides comprising a basic amino acid. e.g., arginine, and a protease.

This application claims the benefit of U.S. patent application Ser. No. 61/027,584 filed Feb. 11, 2008, and also claims the benefit of U.S. patent application Ser. No. 61/027,442 filed Feb. 9, 2008, and U.S. patent application Ser. Nos. 61/027,432; 61/027,431; 61/027,420; and 61/027,435 all filed Feb. 8, 2008 the contents of which applications are all incorporated herein by reference.

### BACKGROUND OF THE INVENTION

Arginine and other basic amino acids have been proposed for use in oral care and are believed to have significant benefits in combating cavity formation and tooth sensitivity. Without intending to be bound by a particular theory, it is hypothesized that a significant factor in the beneficial effect of arginine is that arginine and other basic amino acids can be metabolized by certain types of bacteria, e.g., S. sanguis which are not cariogenic and which compete with cariogenic bacteria such as S. mutans, for position on the teeth and in the oral cavity. The arginolytic bacteria can use arginine and other basic amino acids to produce ammonia, thereby raising the pH of their environment, while cariogenic bacteria metabolize sugar to produce lactic acid, which tends to lower the plaque pH and demineralize the teeth, ultimately leading to cavities. It is believed that regular use of oral compositions comprising arginine, over time, will lead to a relative increase in the arginolytic bacteria and a relative decrease in the cariogenic bacteria, resulting in a higher plaque pH. It is believed that this pH-raising effect may be mechanistically separate from and complementary to the effect of fluoride in promoting remineralization and strengthening the tooth enamel.

However, combining these basic amino acids with minerals having oral care benefits, e.g., fluoride and calcium, to form an oral care product having acceptable long term stability, however, has proven challenging. In particular, the basic amino acid may raise the pH and facilitate dissociation of calcium ions that can react with fluoride ions to form an insoluble precipitate. Moreover, the higher pH has the potential to cause irritation. At neutral pH or acidic pH, however, a system utilizing arginine bicarbonate (which the art teaches is preferred may release carbon dioxide, leading to bloating and bursting of the containers. Moreover, it might be expected that lowering the pH to neutral or acidic conditions would reduce the efficacy of the formulation because the arginine may form an arginine-insoluble calcium complex that has a poorer affinity for the tooth surface, and moreover that lowering the pH would reduce any effect the formulation might have on buffering cariogenic lactic acid in the mouth.

Thus there is a continuing need to develop compositions and methods to deliver basic amino acids to the oral cavity.

### SUMMARY OF THE INVENTION

The invention thus comprises Composition 1.0, an oral care composition comprising an effective amount of a peptide comprising basic amino acids e.g., arginine, in free or salt form, and a protease which cleaves said peptide when said composition is used the oral cavity of a user.

The compositions of the present invention can promote or improve oral health and/or systemic health, including cardiovascular health, e.g., by reducing potential for systemic infection via the oral tissues.

The formulation optionally further comprises
a. a calcium ion source, e.g., a calcium carbonate or a soluble calcium salt, e.g., calcium chloride
b. a phosphate ion source, e.g., a soluble phosphate salt, e.g., potassium phosphate monobasic or dibasic potassium phosphate,
c. a potassium ion source, e.g., potassium chloride or potassium phosphate monobasic or dibasic potassium phosphate,
d. a fluoride source, e.g., a soluble fluoride salt, e.g., sodium fluoride;
e. a polyol humectant, e.g., selected from glycerol, sugar alcohols (e.g., sorbitol, xylitol) and combinations thereof, and/or
f. a protease inhibitor,
   for example any of the following compositions:
   1.0.1. Composition 1.0 wherein the basic amino acids are arginine, lysine, citrullene, ornithine, creatine, histidine, diaminobutanoic acid, diaminoproprionic acid, salts thereof and/or combinations thereof.
   1.0.2. Composition 1.0 or 1.0.1 wherein the basic amino acids of the peptide have the L-configuration.
   1.0.3. Any the preceding compositions wherein the peptide is from about 5 to about 500 amino acids in length, e.g., about 20 to about 100 amino acids.
   1.0.4. Any of the preceding compositions wherein the peptide is enriched with basic amino acids, e.g., has an average nitrogen content of at least about 1.25, e.g., at least about 1.5, e.g., at least about 2 nitrogen atoms per amino acid residue.
   1.0.5. Any of the preceding compositions wherein the peptide comprises L-arginine.
   1.0.6. Any of the preceding compositions wherein the peptide is partially or wholly in salt form.
   1.0.7. Any of the preceding compositions wherein the basic amino acid is present in an amount corresponding to about 0.1 to about 20%, e.g., about 1 wt. % to about 10 wt. % of the total composition weight when the composition is used in the oral cavity, the weight of the basic amino acid being calculated as free base form.
   1.0.8. Composition 1.0.7 wherein the basic amino acid is present in an amount of about 7.5 wt. % of the total composition weight.
   1.0.9. Composition 1.0.7 wherein the basic amino acid is present in an amount of about 5 wt. % of the total composition weight.
   1.0.10. Composition 1.0.7 wherein the basic amino acid is present in an amount of about 3.75 wt. % of the total composition weight.
   1.0.11. Composition 1.0.7 wherein the basic amino acid is present in an amount of about 1.5 wt. % of the total composition weight.
   1.0.12. Any of the preceding compositions wherein the protease is a non-specific protease.
   1.0.13. Any of compositions 1.0-1.0.11 wherein the protease is a specific protease.
   1.0.14. Compositions 1.0.13 wherein the protease is trypsin.
   1.0.15. Any of the preceding compositions wherein the protease is papain.
   1.0.16. Any of the preceding compositions wherein the protease inhibitor is serpin.
   1.0.17. Any of the preceding, compositions wherein the fluoride salt is stannous fluoride, sodium fluoride, potassium fluoride, sodium monofluorophosphate, sodium fluorosilicate, ammonium fluorosilicate, amine fluoride (e.g., N′-octadecyltrimethylendiamine-N,N,N′-tris(2-ethanol)-dihydrofluoride), ammonium fluoride, titanium fluoride, hexafluorosultate, and combinations thereof.
   1.0.18. Any of the preceding compositions wherein the fluoride salt is a fluorophosphate.
   1.0.19. Any of the preceding composition wherein the fluoride salt is sodium monofluorophosphate.
   1.0.20. Any of the preceding compositions wherein the fluoride salt is sodium fluoride.
   1.0.21. Any of the preceding compositions wherein the fluoride salt is present in an amount of about 0.01 wt. % to about 2 wt. % of the total composition weight.
   1.0.22. Any of the preceding compositions wherein the fluoride salt provides fluoride ion in an amount of about 0.1 to about 0.2 wt. % of the total composition weight.
   1.0.23. Any of the preceding compositions wherein the soluble fluoride salt provides fluoride ion in an amount of from about 50 to about 25,000 ppm.
   1.0.24. Any of the preceding compositions which is a mouthwash having about 100 to about 250 ppm available fluoride ion.
   1.0.25. Any of the preceding compositions which is a dentifrice having about 750 to about 2000 ppm available fluoride ion.
   1.0.26. Any of the preceding compositions wherein the composition further comprises about 750 to about 2000 ppm fluoride ion.
   1.0.27. Any of the preceding compositions wherein the composition further comprises about 1000 to about 1500 ppm fluoride ion.
   1.0.28. Any of the preceding compositions wherein the composition further comprises about 1450 ppm fluoride ion.
   1.0.29. Any of the preceding compositions wherein the pH is about 6 to about 9, e.g., about 6.5 to about 7.4 or about 7.5 to about 9.
   1.0.30. Any of the preceding compositions wherein the pH is about 6.5 to about 7.4.
   1.0.31. Any of the preceding compositions wherein the pH is about 6.8 to about 7.2.
   1.0.32. Any of the preceding compositions wherein the pH is approximately neutral.
   1.0.33. Any of the preceding compositions further comprising an abrasive or particulate.
   1.0.34. The immediately preceding composition wherein the adhesive or particulate is selected from bicarbonate, calcium phosphate (e.g., dicalcium phosphate dihydrate calcium sulfate, precipitated calcium carbonate, silica (e.g., hydrated silica), iron oxide, aluminum oxide, perlite, plastic particles, (e.g., polyethylene), and combinations thereof.
   1.0.35. The immediate preceding composition wherein the abrasive or particulate is selected from a calcium phosphate (e.g., dicalcium phosphate dihydrate), calcium sulfate, precipitated calcium carbonate, silica (e.g., hydrated silica), and combinations thereof.
   1.0.36. Any of the preceding compositions further comprising an abrasive in an amount of about 15 wt. % to about 70 wt. % of the total composition weight.
   1.0.37. Any of the preceding compositions further comprising a small particle abrasive fraction of at least about 5% having a d50 of<5 micrometers.
   1.0.38. Any of the preceding compositions having an RDA of less than about 150, e.g., about 40 to about 140.
   1.0.39. Any of the preceding compositions wherein the anionic surfactant is selected from
a. water-soluble salts of higher fatty acid monoglyceride monosulfates (e.g., the sodium salt of the monosulfated monoglyceride of hydrogenated coconut oil fatty acids such as sodium N-methyl N-cocoyl taurate, sodium cocomo-glyceride sulfate),
b. higher alkyl sulfates, e.g., sodium lauryl sulfate,
c. higher alkyl-ether sulfates, e.g., of formula CH₂(CH₂)ₘCH₂(OCH₂CH₂)ₙOSO₃X, wherein m is 6-16, e.g., 10, n is 1-6, g., 2, 3 or 4, and X is Na or K (for example sodium laureth-2 sulfate (CH₃(CH₂)₁₀CH₂(OCH₂CH₂)₂OSO₃Na)),
d. higher alkyl aryl sulfonates (such as sodium dodecyl benzene sulfonate (sodium lauryl benzene sulfonate)),
e. higher alkyl sulfoacetates (such as sodium lauryl sulfoacetate (dodecyl sodium sulfoacetate), higher fatty acid esters of 1,2 dihydroxy propane sulfonate, sulfocolaurate (N-2-ethyl laurate potassium sulfoacetamide) and sodium lauryl sarcosinate).
f. and mixtures thereof.
   By “higher alkyl” is meant, C₆₋₃₀ alkyl. In particular embodiments, the anionic surfactant is selected from sodium lauryl sulfate and sodium ether lauryl sulfate.
   1.0.40. Any of the preceding compositions wherein the anionic surfactant is selected from sodium lauryl sulfate, sodium ether lauryl sulfate, and mixtures thereof.
   1.0.41. Any of the preceding compositions wherein the anionic surfactant is present in an amount of from about 0.3% to about 4.5% by weight.
   1.0.42. Any of the preceding compositions additionally comprising surfactants selected from cationic, zwitterionic, and nonionic surfactants, and mixtures thereof.
   1.0.43. Any of the preceding compositions further comprising at least one humectant.
   1.0.44. Any of the preceding compositions further comprising at least one humectant selected from glycerin, sorbitol, xylitol, and combinations thereof.
   1.0.45. Any of the preceding compositions further comprising xylitol.
   1.0.46. Any of the preceding compositions further comprising at least one polymer.
   1.0.47. Any of the preceding compositions further comprising at least one polymer selected from polyethylene glycols, polyvinylmethyl ether maleic acid copolymers, polysaccharides (e.g., cellulose derivatives, for example carboxymethyl cellulose, or polysaccharide gums, for example xanthan gum or carrageenan gum), and combinations thereof.
   1.0.48. Any of the preceding compositions further comprising gum strips or fragments.
   1.0.49. Any of the preceding compositions further comprising flavoring, fragrance and/or coloring.
   1.0.50. Any of the preceding compositions further comprising water.
   1.0.51. Any of the preceding compositions further comprising an antibacterial agent selected from halogenated diphenyl ether (e.g. triclosan), herbal extracts and essential oils (e.g., rosemary extract, tea extract, magnolia extract, thymol, menthol, eucalyptol, geraniol, carvacrol, citral, hinokitol, catechol, methyl salicylate, epigallocatechin gallate, epigallocatechin, gallic acid, miswak extract, sea-buckthorn extract), bisguanide antiseptics (e.g., chlorhexidine, alexidine or octenidine), quaternary ammonium compounds (e.g., cetylpyridinium chloride (CPC), benzalkonium chloride, tetradecylpyridinium chloride (TPC), N-tetradecyl-4-ethylpyridinium chloride (TDEPC)), phenolic antiseptics, hexetidine, octenidine, sanguinarine, povidone iodine, delmopinol, salifluor, metal ions (e.g., zinc salts, for example, zinc citrate, stannous salts, copper salts, iron salts), sanguinarine, propolis and oxygenating agents (e.g., hydrogen peroxide, buffered sodium peroxyborate or peroxycarbonate), phthalic acid and its salts, monoperthalic acid and its salts and esters, ascorbyl stearate, oleoyl sarcosine, alkyl sulfate, dioctyl sulfosuccinate, salicylanilide, domiphen bromide, delmopinol, octapinol and other piperidino derivatives, nicin preparations, chlorite salts; and mixtures of any of the foregoing.
   1.0.52. Any of the preceding compositions further comprising an anti-inflammatory compound, e.g., an inhibitor of at least one of host pro-inflammatory factors selected from matrix metalloproteinases (MMP's), cyclooxygenases (COX), PGE₂, interleukin 1 (IL-1), IL-1β converting enzyme (ICE), transforming growth factor β1 (TGF-β1), inducible nitric oxide synthase (iNOS), hyaluronidase, cathepsins, nuclear factor kappa B (NF-κB), and IL-1 Receptor Associated Kinase (IRAK), e.g. selected from aspirin, ketorolac, flurbiprofen, ibuprofen, naproxen, indomethacin, aspirin, ketoprofen, piroxicam, meclolenamic acid, nordihydoguaiaretic acid, and mixtures thereof.
   1.0.53. Any of the preceding compositions further comprising an antioxidant, e.g., selected from the group consisting of Co-enzyme Q10, PQQ, Vitamin C, Vitamin E, Vitamin A, anethole-dithiothione, and mixtures thereof.
   1.0.54. Any of the preceding compositions wherein the anti-microbial is poorly soluble.
   1.0.55. Any of the preceding compositions further comprising triclosan.
   1.0.56. Any of the preceding compositions further comprising triclosan and xylitol.
   1.0.57. Any of the preceding compositions further comprising triclosan, xylitol, and precipitated calcium carbonate.
   1.0.58. Any of the preceding compositions further comprising an antibacterial agent in an amount of about 0.01 to about 5 wt. % of the total composition weight.
   1.0.59. Any of the preceding compositions further comprising triclosan in an amount of about 0.01 to about 1 wt. percent of the total composition weight.
   1.0.60. Any of the preceding compositions further comprising triclosan in an amount of about 0.3% of the total composition weight.
   1.0.61. Any of the preceding compositions further comprising a whitening agent.
   1.0.62. Any of the preceding compositions further comprising a whitening agent selected from a whitening active selected from the group consisting of peroxides, metal chlorites, perborates, perearbonates, peroxyacids, hypochlorites, and combinations thereof
   1.0.63. Any of the preceding compositions further comprising hydrogen peroxide or a hydrogen peroxide source, e.g., urea peroxide or a peroxide salt or complex (e.g., such as peroxyphosphate, peroxycarbonate perborate, peroxysilicate, or persulphate salts: for example calcium peroxyphosphate, sodium perborate, sodium carbonate peroxide, sodium peroxyphosphate. and potassium persulfate).
   1.0.64. Any of the preceding compositions further comprising an agent that interferes with or prevents bacterial attachment, e.g., solbrol or chitosan.
   1.0.65. Any of the preceding compositions further comprising a source of calcium and phosphate selected from (i) calcium-glass complexes, e.g., calcium sodium phosphosilicates, and (ii) calcium-protein complexes, e.g., casein phosphopeptide-amorphous calcium phosphate.
   1.0.66. Any of the preceding compositions further comprising a soluble calcium salt, e.g., selected from calcium sulfate, calcium chloride, calcium nitrate, calcium acetate, calcium lactate, and combinations thereof.
   1.0.67. Any of the preceding compositions further comprising a physiologically acceptable potassium salt, e.g., potassium nitrate or potassium chloride, in an amount effective to reduce dentinal sensitivity.
   1.0.68. Any of the preceding compositions further comprising from about 0.1% to about 7.5% of a physiologically acceptable potassium salt, e.g., potassium nitrate and/or potassium chloride.
   1.0.69. Any of the preceding compositions which is a toothpaste comprising an arginine salt, e.g., arginine hydrochloride, arginine phosphate or arginine bicarbonate; triclosan; an anionic surfactant, e.g., sodium lauryl sulfate; and a soluble fluoride salt, e.g., sodium monafluorophosphate or sodium fluoride.

1.0.70. Any of the preceding compositions effective upon application to the oral cavity, e.g., with brushing, to (i) reduce or inhibit formation of dental caries, (ii) reduce, repair or inhibit pre-carious lesions of the enamel, e.g., as detected by quantitative light-induced fluorescence (QLF) or electrical caries measurement (ECM), (iii) reduce or inhibit demineralization and promote remineralization of the teeth, (iv) reduce hypersensitivity of the teeth, (v) reduce or inhibit gingivitis, (vi) promote healing of sores or cuts in the mouth, (vii) reduce levels of acid producing bacteria, (viii) to increase relative levels of arginolytic bacteria, (ix) inhibit microbial biofilm formation in the oral cavity, (x) raise and/or maintain plaque pH at levels of at least pH 5.5 following sugar challenge, (xi) reduce plaque accumulation, (xii) treat, relieve or reduce dry mouth,(xiii) clean the teeth and oral cavity (xiv) reduce erosion, (xv) whiten teeth, (xvi) immunize the teeth against cariogenic bacteria; and/or (xvii) promote systemic health, including cardiovascular health, e.g., by reducing potential for systemic infection via the oral tissues.

1.0.71. A composition obtained or obtainable by combining the ingredients as set forth in any of the preceding compositions.
1.0.72. Any of the preceding compositions in a form selected from mouthrinse, toothpaste, tooth gel, tooth powder, non-abrasive gel, mousse, foam, mouth spray, lozenge, oral tablet, dental implement, and pet care product.
1.0.73. Any of the preceding compositions wherein the composition is toothpaste.
1.0.74. Any of the preceding compositions wherein the composition is a toothpaste optionally further comprising one or more of one or more of water, abrasives, surfactants, foaming agents, vitamins, polymers, enzymes, humectants. thickeners, antimicrobial agents, preservatives, flavorings, colorings and/or combinations thereof.
1.0.75. Any of the preceding compositions 1.0-1.0.73 wherein the composition is a mouthwash.
1.0.76. Any of the preceding compositions 1.0-1.0.73 wherein the composition is a chewing gum.
1.0.77. Any of the preceding compositions further comprising a breath freshener, fragrance or flavoring.
1.0.78. Any of the preceding compositions wherein the protein is soy protein or soy protein derivative.
1.0.79. Any of the preceding composition wherein the protein is ground nut protein, or ground nut protein derivative.
1.0.80. Any of the preceding compositions wherein the peptide is derived by partially hydrolyzing or partially digesting a protein and enriching mixture of peptides for basic amino acids arginine.
1.0.81. Any of the preceding compositions wherein the peptide provides a basic pH to an aqueous solution, e.g., a pH of at least about 7.5, e.g., at least about 8, e.g., about 8 to about 10.

The present invention also includes Method 2.0, comprising applying any of the preceding compositions e to the oral cavity, e.g., with brushing, to (i) reduce or inhibit formation of dental caries, (ii) reduce, repair or inhibit pre-carious lesions of the enamel, e.g., as detected by quantitative light-induced fluorescence (QLF) or electrical caries measurement (ECM), (iii) reduce or inhibit demineralization and promote remineralization of the teeth, (iv) reduce hypersensitivity of the teeth, (v) reduce or inhibit gingivitis, (vi) promote healing of sores or cuts in the mouth, (vii) reduce levels of acid producing bacteria, (viii) to increase relative levels of arginolytic bacteria, (ix) inhibit microbial biofilm formation in the oral cavity, (x) raise and/or maintain plaque pH at levels of at least pH 5.5 following sugar challenge, (xi) reduce plaque accumulation, (xii) reduce dry mouth. (xiii) clean the teeth and oral cavity (xiv) reduce erosion, (xv) whiten teeth, and/or (xvi) immunize the teeth against cariogenic bacteria comprising: introducing into the oral cavity to a patient in need thereof an oral care according to any one of compositions 1.0-1.0.78.

Additional embodiments of the present invention also include the following methods:

2.1 Of method 2.0, wherein the protease hydrolyzes the peptide when introduced into the oral cavity.
2.2 Of method 2.0 or 2.1 wherein the protease inhibitor is inactivated or diluted when the composition is introduced into the oral cavity
2.3 Of methods 2.0 or 2.2 wherein the composition comprises at least about 7.5% arginine.
2.4 Of methods 2.0-2.3 wherein the composition is a dentifrice.
2.5 Of methods 2.0-2.4 wherein the composition is a toothpaste.
2.6 Of methods 2.0-2.5 wherein the composition is a gel.
2.7 Of methods 2.0-2.6 wherein the composition is applied in the oral cavity with a tooth brush.
2.8 Of methods 2.0-2.3 wherein the composition is a mouth wash.

Levels of active ingredients will vary based on the nature of the delivery system and the particular active. For example, the protein comprising basic amino acids may be present at levels from, e.g., about 0.1 to about 20 wt % (expressed as weight of free base e.g., about 0.1 to about 3 wt % for a mouthrinse, about 1 to about 10 wt % for a consumer toothpaste or about 7 to about 20 wt % for a professional or prescription treatment product. Fluoride may be present at levels of, e.g., about 25 to about 25,000 ppm, for example about 25 to about 250 ppm for a mouthrinse, about 750 to about 2,000 ppm for a consumer toothpaste, or about 2,000 to about 25,000 ppm for a professional or prescription treatment product. Levels of antibacterial will vary similarly, with levels used in toothpaste being about 5 to about 15 times greater than used in mouthrinse. For example, a triclosan mouthrinse may contain, e.g., about 0.03 wt % triclosan while a triclosan toothpaste may contain about 0.3 wt % triclosan.

Other embodiments of the present invention will be apparent to one of skill in the art.

### DETAILED DESCRIPTION OF THE INVENTION

Peptides and their formation are known in the art and are short polymers of amino acids. Peptides of the present invention may he, e.g., from about 5 to about 500 amino acids in length, preferably, wherein a majority of the amino acids are basic amino acids, more preferably, all of the amino acids are basic amino acids, e.g., wherein the ratio of nitrogen atoms to amino acid residues exceeds about 0.25, e.g., about 1.5, e.g., about 2; e.g., wherein the amino acid has a net positive charge, e.g., provides a basic pH to a solution, e.g., a pH of greater than about 7.5, e.g., greater than about 8.

Large proteins, e.g., soy or ground nut protein, may be hydrolyzed or digested into smaller proteins, and the fragments rich in basic amino acids, especially arginine, may be separated. For example, peptides comprising basic amino acids tend to be somewhat more soluble at higher pH than less basic peptides. Methods of obtaining arginine-rich fractions are described, e.g., in U.S. Pat. No. 7,091,001 and separation of arginine from other amino acids by taking advantage of relative solubility at different pH has been described as far back as 1900. See, e.g., Kossel, A., and Kutscher, F., Z. Physiol. Chem., 1900, xxxi, 165. Thus arginine-enriched protein fractions are available to one of skill in the art.

Proteases are known in the art, and include a class of enzymes which degrades peptides by hydrolyzing peptide bonds. Proteases may be specific or non-specific proteases, either of which may be used in the present invention, depending on the particular peptide.

Non-specific proteases are known in the art and may hydrolyze most or all peptide bonds, irrespective of the amino acid. Specific proteases only hydrolyze peptide bonds of specific amino acids, depending on the amino acid sequence. Thus, specific proteases for use in the compositions of the present invention are dependent upon the particular peptide sequence. For example, trypsin cleaves proteins at, the carboxyl side of lysine and arginine, and thus would he suitable for use with polypeptides of lysine, arginine, and lysine and arginine.

Preferred proteases Include endopeptidases which cleaves the polypeptide within the polypeptide chain rather than at the terminal amino acids.

The compositions of the present invention may also comprise an effective amount of one or more protease inhibitors, which are known in the art. Selection of particular protease inhibitors will be dependent upon the specific protease incorporated into the composition. For example, when trypsin is incorporated as a protease, serpin may be used as a protease inhibitor. Preferably, protease inhibitor in concentrations which inhibit protease activity while compositions of the present invention are not used in the oral cavity, e.g., during manufacture, processing, storage, or shipping, but become inactive, e.g., diluted, when the compositions are used in the oral cavity such that the protease inhibitor will no longer prevent protease activity.

The composition may comprise useful enzymes which include any of the available proteases, glucanohydrolases, endoglycosidases, amylases, mutanases, lipases and mucinases or compatible mixtures thereof. In certain embodiments, the enzyme is a protease, dextranase, endoglycosidase and mutanase. In another embodiment, the enzyme is papain, endoglycosidase or a mixture of dextranase and mutanase. Additional enzymes suitable for use in the present invention are disclosed in U.S. Pat. No. 5,000,939 to Dring et al., U.S. Pat. No. 4,992,420; U.S. Pat. No. 4,355,022; U.S. Pat. No. 4,154,815; U.S. Pat. No. 4,058,595; U.S. Pat. No. 3,991,177; and U.S. Pat. No. 3,696,191 all incorporated herein by reference. An enzyme or a mixture of several compatible enzymes in the current invention constitutes about 0.002% to about 2.0% in one embodiment or about 0.05% to about 1.5% in another embodiment or in yet another embodiment about 0.1% to about 0.5%.

The peptides of the present invention comprise basic amino acids, which include not only naturally occurring basic amino acids, such as arginine, lysine, and histidine, but also any basic amino acids having a carboxyl group and an amino group in the molecule, which are water-soluble and provide an aqueous solution with a pH of about 7 or greater. Accordingly, basic amino acids include, but are not limited to, arginine, lysine, citrullene, ornithine, creatine, histidine, diaminobutanoic acid, diaminoproprionic acid, or combinations thereof. In a particular embodiment, the basic amino acids are selected from arginine, citrullene, and ornithine. In certain embodiments, the basic amino acid is arginine, for example, L-arginine, or a salt thereof.

The compositions of the invention are intended for topical use is the and so peptide salts for use in the present invention should be safe for such use, in the amounts and concentrations provided. Suitable salts include salts known in the art to be pharmaceutically acceptable salts are generally considered to be physiologically acceptable in the amounts and concentrations provided. Physiologically acceptable salts include those derived from pharmaceutically acceptable inorganic or organic acids or bases, for example acid addition salts formed by acids which form a physiological acceptable anion, e.g., hydrochloride or bromide salt, and base addition salts formed by bases which form a physiologically acceptable cation, for example those derived from alkali metals such as potassium and sodium or alkaline earth metals such as calcium and magnesium. Physiologically acceptable salts may be obtained using standard procedures known in the art, for example, by reacting a sufficiently basic compound such as an amine with a suitable acid affording a physiologically acceptable anion.

Concentrations of arginine in oral care compositions for anti-caries effect may be about 1.5%. Higher concentrations of arginine may be utilized for sensitive tooth relief, e.g., from about 3.75% to about 7.50% arginine, as the formulations physically occlude open dentinal tubules (pathways to pain), and provide effective pain relief. Without being bound by theory, it is hypothesized that even higher levels of arginine, e.g., greater than about 7.50%, that is, from about 7.50% to about 25%, from about 8.0% to about 20%, from about 9% to about 15%, or about 10% coat teeth, gums, and/or the oral cavity, leaving a perception that the mouth has been moisturized or hydrated.

Compositions of the present invention comprise an effective amount of a peptide comprising basic amino acids. An effective amount is an amount effective to achieve the benefits of a basic amino acid, e.g., arginine, in the oral cavity following hydrolysis of the peptide by the protease. Thus it will be realized that an effective amount of the peptide will be dependent on the amount of protease present in the composition.

Compositions of the present invention comprise an effective amount of a protease which hydrolyzes the peptide. Thus it will be realized that an effective amount of the protease will be dependent on the amount of peptide present in the composition, and the particular protease selected. In compositions comprising a peptide, protease, and protease inhibitor, the effective amount of the protease may be dependent upon the levels of peptide and the protease inhibitor.

Compositions of the present invention may comprise an effective amount of a protease inhibitor which inhibits protease hydrolysis of the peptide until the composition is released in the oral cavity. Effective amounts of the protease inhibitor will depend not only on the amounts of protease, but the type of protease, and the type of protease inhibitor.

One of skill in the art may determine effective amounts of a peptide, protease, and protease inhibitor. Compositions comprising varying amounts of such may be created, and the basic amino acid content of such compositions may be assayed before use, and when released in the oral cavity.

Compositions of the present invention may be in the form of a dentifrice comprising additional ingredients selected from one or more of water, abrasives, surfactants, foaming agents, vitamins, polymers, enzymes, humectants, thickeners, antimicrobial agents, preservatives, flavorings, colorings and/or combinations thereof.

The oral care compositions may further include one or more fluoride ion sources, e.g., soluble fluoride salts. A wide variety of fluoride ion-yielding materials can be employed as sources of soluble fluoride in the present compositions, and such materials are known to those of skill in the art. Examples of suitable fluoride ion-yielding materials are found in U.S. Pat. No. 3,535,421, to Briner et al.; U.S. Pat. No. 4,885,155, to Parran, Jr. et al. and U.S. Pat. No. 3,678,154, to Widder et al., incorporated herein by reference.

Representative fluoride ion sources include, but are not limited to, stannous fluoride, sodium fluoride, potassium fluoride, sodium monofluorophosphate, sodium fluorosilicate, ammonium fluorosilicate, amine fluoride, ammonium fluoride, and combinations thereof. In certain embodiments the fluoride ion source includes stannous fluoride, sodium fluoride, sodium monofluorophosphate as well as mixtures thereof.

In certain embodiments, the oral care composition of the invention may also contain a source of fluoride ions or fluorine-providing ingredient in amounts sufficient to supply about 25 ppm to 25,000 ppm of fluoride ions, generally at least about 500 ppm, e.g., about 500 to about 2000 ppm, e.g., about 1000 to about 1600 ppm, e.g., about 1450 ppm. The appropriate level of fluoride will depend on the particular application. A mouthwash, for example, would typically have about 100 to about 250 ppm fluoride. A toothpaste for general consumer use would typically have about 1000 to about 1500 ppm, with pediatric toothpaste having somewhat less. A dentifrice or coating for professional application could have as much as 5,000 or even 25.000 ppm fluoride.

Fluoride ion sources may be added. to the compositions or the invention at a level of about 0.01 wt. to about 10 wt. % in one embodiment or about 0.03 wt. % to about 5 wt. %, and in another embodiment about 0.1 wt. % to about 1 wt. % by weight of the composition in another embodiment. Weights of fluoride salts to provide the appropriate level of fluoride ion will obviously vary based on the weight of the counter ion in the salt.

The Compositions of the invention may comprise a calcium phosphate abrasive, e.g., tricalcium phosphate (Ca₃(PO₄)₂), hydroxyapatite (Ca₁₀)PO₄)₆(OH)₂), or dicalcium phosphate dihydrate (CaHPO₄·2H₂O, also sometimes referred to herein as DiCal) or calcium pyrophosphate.

The compositions may include one or more additional particulate materials, for example silica abrasives such as precipitated silicas having a mean particle size of up to about 20 microns, such as Zeodent 115®, marketed by J. M. Huber. Other useful abrasives also include sodium metaphosphate, potassium metaphosphate, aluminum silicate, calcined alumina, bentonite or other siliceous materials, or combinations thereof.

The silica abrasive polishing materials useful herein, as well as the other abrasives, generally have an average particle size ranging between about 0.1 and about 30 microns, about between 5 and about 15 microns. The silica abrasives can be from precipitated silica or silica gels, such as the silica xerogels described in U.S. Pat. No. 3,538,230, to Pader at al. and U.S. Pat. No. 3,862,307, to Digiulio, both incorporated herein by reference. Particular silica xerogels are marketed under the trade name Syloid® by the W. R. Grace & Co., Davison Chemical Division. The precipitated silica materials include those marketed by the J. M. Huber Corp. under the trade name Zeodent®, including the silica carrying the designation Zeodent 115 and 119. These silica abrasives are described in U.S. Pat. No. 4,340,583, to Wason, incorporated herein by reference.

In certain embodiments, abrasive materials useful in the practice of the oral care compositions in accordance with the invention include silica gels and precipitated amorphous silica having an oil absorption value of about less than 100 cc/100 g silica and in the range of about 45 cc/100 g to about 70 cc/100 g silica. Oil absorption values are measured using the ASTA Rub-Out Method D281. In certain embodiments, the silicas are colloidal particles having an average particle size of about 3 microns to about 12 microns, and about 5 to about 10 microns

In particular embodiments, the particulate or abrasive materials comprise a large fraction of very small particles, e.g., having a d50 less than about 5 microns, for example small particle silica (SPS) having a d50 of about 3 to about 4 microns, for example Sorbosil AC43® (Ineos). Such small particles are particularly useful in formulations targeted at reducing hypersensitivity. The small particle component may be present in combination and larger particle abrasive. In certain embodiments, for example, the formulation comprises about 3 to about 8% SPS and about 25 to about 45% of a conventional abrasive.

Low oil absorption silica abrasives particularly useful in the practice of the invention are marketed under the trade designation Sylodent XWA® by Davison Chemical Division of W.R. Grace & Co., Baltimore, Md. 21203. Sylodent 650 XWA®, a silica hydrogel composed of particles of colloidal silica having a water content of about 29% by weight averaging about 7 to about 10 microns in diameter, and an oil absorption of less than about 70 cc/100 g of silica is an example of a low oil absorption silica abrasive useful in the practice of the present invention. The abrasive is present in the oral care composition of the present invention at a concentration of about 10 to about 60% by weight, in other embodiment about 20 to about 45% by weight, and in another embodiment about 30 to about 50% by weight.

The oral care compositions of the invention also may include an agent to increase the amount of foam that is produced when the oral cavity is brushed. Such agents are known to those of skill in the art. Illustrative examples of agents that increase the amount of foam include, but are not limited to polyoxyethylene and certain polymers including, but not limited to, alginate polymers.

The polyoxyethylene may increase the amount of foam and the thickness of the foam generated by the oral care carrier component of the present invention. Polyoxyethylene is also commonly known as polyethylene glycol (“PEG”) or polyethylene oxide. The polyoxyethylenes suitable for this invention will have a molecular weight of about 200,000 to about 7,000,000. In one embodiment the molecular weight will be about 600,000 to about 2,000,000 and in another embodiment about 800,000 to about 1,000,000. Polyox® is the trade name for the high molecular weight polyoxyethylene produced by Union Carbide.

The polyoxyethylene may be present in an amount of about 1% to about 90%, in one embodiment about 5% to about 50% and in another embodiment about 10% to about 20% by weight of the oral care carrier component of the oral care compositions of the present invention. The dosage of foaming agent in the oral care composition (i.e., a single dose) is about 0.01 to about 0.9% by weight, about 0.05 to about 0.5% by weight, and in another embodiment about 0.1 to about 0.2% by weight.

Another agent optionally included in the oral care composition of the invention is a surfactant or a mixture of compatible surfactants. Suitable surfactants are those which are reasonably stable throughout a wide pH range, for example, anionic, cationic, nonionic or zwitterionic surfactants. Suitable surfactants are described more fully, for example, in U.S. Pat. No. 3,959,458, to Agricota et al.:, U.S. Pat. No. 3,937,807, to Haefele; and U.S. Pat. No. 4,051,234, to Gieske et al., which are incorporated herein by reference. A preferred surfactant is sodium lauryl sulfate.

The surfactant or mixtures of compatible surfactants can be present in the compositions of the present invention in about 0.1% to about 5.0%, in another embodiment about 0.3% to about 3.0% and in another embodiment about 0.5% to about 2.0% by weight of the total composition.

The oral care compositions of the invention may also include a flavoring agent. Flavoring agents which are used in the practice of the present invention are known by those of skill in the art, and may include essential oils as well as various flavoring aldehydes, esters, alcohols, and similar materials. The flavoring agent is incorporated in the oral composition at a concentration of about 0.1 to about 5% by weight and about 0.5 to about 1.5% by weight. The dosage of flavoring agent in the individual oral care composition dosage (i.e., a single dose) is about 0.001 to about 0.05% by weight and in another embodiment about 0.005 to about 0.015% by weight.

The oral care compositions and methods of the invention also may optionally include one or more chelating agents able to complex calcium found in the cell walls of the bacteria. Binding of this calcium weakens the bacterial cell wall and augments bacterial lysis. Chelating agents are well known by those of skill in the art, e.g., soluble pyrophosphates, either in hydrated or unhydrated forms. An effective amount of pyrophosphate salt useful in the present composition is generally enough to provide at least about 1.0 wt % pyrophosphate ions, about 1.5 wt. % to about 6 wt. %, about 3.5 wt. % to about 6 wt. % of such ions.

The oral care compositions or methods of the invention also optionally include one or more polymers, which are known by those of skill in the art. Such polymers may include polyethylene glycols, polyvinylmethyl ether maleic acid copolymers, polysaccharides cellulose derivatives, for example carboxymethyl cellulose, or polysaccharide gums, for example xanthan gum or carrageenan gum). Polymers suitable for use may include Gantrez AN 139(M.W. 500,000), AN 119 (M.W. 250,000) and S-97 Pharmaceutical Grade (M.W. 70,000), of GAF Chemicals Corporation. Suitable polymers may also include homopolymers of substituted acrylamides and/or homopolymers of unsaturated sulfonic acids and salts thereof, in particular where polymers are based on unsaturated sulfonic acids selected from acrylamidoalykane sulfonic acids such as 2-acrylamide 2 methylpropane sulfonic acid having a molecular weight of about 1,000 to about 2,000,000 described in U.S. Pat. No. 4,842 847, Jun. 27, 1989 to Zahid, incorporated herein by reference. Another useful class of polymeric agents includes polyamino acids, particularly those containing proportions of anionic surface-active amino adds such as aspartic acid, glutamic acid and phosphoserine, as disclosed in U.S. Pat. No. 4,866,161 Sikes et al., incorporated herein by reference.

The compositions and methods of the present invention may also comprise thickening material to provide a desirable consistency or to stabilize or enhance the performance of the formulation. Such thickening materials are known by those of skill in the art, e.g., carboxyvinyl polymers, carrageenan, hydroxyethyl cellulose and water soluble salts of cellulose ethers such as sodium carboxymethyl cellulose and sodium carboxymethyl hydroxyethyl cellulose. Natural gums such as karaya, gum arabic, and gum tragacanth can also be incorporated. Colloidal magnesium aluminum silicate or finely divided silica can be used as component of the thickening composition to further improve the composition's texture. In certain embodiments, thickening agents in an amount of about 0.5% to about 5.0% by weight of the total composition are used.

The compositions and methods of the present invention may also optionally include one or more enzymes. Useful enzymes include those known by those of skill in the art, and may include proteases, glucanohydrolases, endoglycosidases, amylases, mutanases, lipases and mucinases or compatible mixtures thereof. Enzymes suitable for use in the present invention are disclosed in U.S. Pat. No. 5,000,939 to Dring et al., U.S. Pat. No. 4,992,420; U.S. Pat. No. 4,355,022; U.S. Pat. No. 4,154,815; U.S. Pat. No. 4,058,595; U.S. Pat. No. 3.991,177; and U.S. Pat. No. 3,696,191 all incorporated herein by reference. An enzyme of a mixture of several compatible enzymes in the current invention constitutes about 0.002% to about 2.0% in one embodiment or about 0.05% to about 1.5% in another embodiment or in yet another embodiment about 0.1% to about 0.5%.

Water may also be present in the oral compositions of the invention. Water, employed in the preparation of commercial oral compositions is preferably deionized and free of organic impurities. Water commonly makes up the balance of the compositions, and includes the free water which is added plus that amount which is introduced with other materials such as with sorbitol or any components of the invention.

The present invention may comprise humectant to prevent the composition from hardening upon exposure to air, and to aid in the hydration of the mouth. Certain humectants can also impart desirable sweetness or flavor to dentifrice compositions. The humectant, on a pure humectant basis, generally includes about 15% to about one embodiment or about 30% to about 65% in another embodiment by weight of the dentifrice composition.

Suitable humectants include edible polyhydric alcohols such as glycerine, sorbitol, xylitol, propylene glycol as well as other polyols and mixtures of these humectants. Mixtures of glycerine and sorbitol may be used in certain embodiments as the humectant component of the toothpaste compositions herein.

In addition to the above described components. the embodiments of this invention can contain a variety of optional dentifrice ingredients some of which are described below. Optional ingredients include, for example, but are not limited to, adhesives, sudsing agents, flavoring agents, sweetening agents, additional antiplaque agents, abrasives, and coloring agents. These and other optional components are further described in U.S. Pat. No. 5,004,597, to Majeti; U.S. Pat. No. 3,959,458 to Agricola et al. and U.S. Pat. No. 3,937.807, to Haefele, all being incorporated herein by reference.

The compositions and methods according to the invention are useful to a method to treat dry mouth, and optionally protect the teeth by facilitating repair and remineralization, in particular to reduce or inhibit formation of dental caries, reduce or inhibit demineralization and promote remineralization of the teeth, reduce hypersensitivity of the teeth, and reduce, repair or inhibit pre-carious lesions of the enamel, e.g., as detected by quantitative light-induced fluorescence (OLE) or electrical conductance measurement (ECM). Quantitative light-induced fluorescence is a visible light system that permits early detection of pre-caries lesions in the enamel. Normal teeth fluoresce in visible light; demineralized teeth do not or do so only to a lesser degree. The area of demineralization can be quantified and its progress monitored. Electrical conductance measurement exploits the fact that the fluid-filled tubules exposed upon demineralization and erosion of the enamel conduct electricity. An increase in the conductance of the patient's teeth therefore may indicate demineralization. The Compositions of the Invention are thus useful in a method to reduce pre-carious lesions of the enamel (as measured by QLF or ECM) relative to a composition lacking effective amounts of fluorine and/or arginine.

Enhancing oral health also provides benefits in systemic health, as the oral tissues can he gateways for systemic infections. Good oral health is associated with systemic health, including cardiovascular health. The compositions and methods of the invention provide particular benefits because basic amino acids, especially arginine, are sources of nitrogen which supply NO synthesis pathways and thus enhance microcirculation in the oral tissues that is less favorable to Heliobacter, which is associated with gastric ulcers. Arginine in particular is required for high expression of specific immune cell receptors, for example T-cell receptors, so that arginine can enhance an effective immune response. The compositions and methods of the invention are thus useful to enhance systemic health, including cardiovascular health. Providing a less acidic oral environment is also helpful in reducing gastric distress and creates an environment less favourable to Heliobacter, which is associated with gastric ulcers. Arginine in particular is required for high expression of specific immune. cell receptors, for example T-cell receptors, so that arginine can enhance an effective immune response. The compositions and methods of the invention are thus useful to enhance systemic health, including cardiovascular health.

The compositions and methods according to the invention can he incorporated into oral compositions for the care of the mouth and teeth such as toothpastes, transparent pastes, gels, mouth rinses, sprays and chewing gum.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls. It is understood that when formulations are described, they may be described in terms of their ingredients, as is common in the art, notwithstanding that these ingredients may react with one another in the actual formulation as it is made, stored and used, and such products are intended to be covered by the formulations described.

The following examples further describe and demonstrate illustrative embodiments within the scope of the present invention. The examples are given solely for illustration and are not to be construed as limitations of this invention as many variations are possible without departing from the spirit and scope thereof. Various modifications of the invention in addition to those shown and described herein should be apparent to those skilled in the art and are intended to fall within the appended claims.

1. An oral care composition comprising:
(i) an effective amount of a peptide or mix of peptides enriched with basic amino acids, and
(ii) a protease which cleaves said peptide and releases a basic amino acid into an oral cavity when said composition is used in the oral cavity of a user.

2. The composition of claim 1 wherein the peptide is from about 5 to about 500 amino acids in length.

3. The composition of claim 1 wherein the composition further comprises one or more of:
a. a calcium ion source,
b. a phosphate ion source,
c. a potassium ion source,
d. a fluoride ion source,
e. a polyol humectant, and
f. a protease inhibitor.

4. The composition of claim 1 wherein at least one of said the basic amino acids is selected from arginine, lysine, citrullene, ornithine, creatine, histidine, diaminobutanoic acid, diaminoproprionic acid, salts thereof and combinations thereof.

5. The composition of claim 1 wherein greater than about 50% of the amino acids in the peptide are basic amino acids.

6. The composition of claim 1 comprising a mixture of peptides derived by partially hydrolyzing or partially digesting a protein and enriching the resulting fragments for peptides comprising basic amino acids.

7. The composition of claim 1 wherein the peptide provides a basic pH to an aqueous solution.

8. The composition of claim 1 wherein the peptide is enriched with arginine.

9. The composition of claim 1 wherein the basic amino acid is present in an amount corresponding to about 0.1 to about 20% of the total composition weight, wherein the weight of the basic amino acid is calculated as based on its free base form.

10. The composition of claim 7 wherein the basic amino acid is present in an amount corresponding to about 1 wt. % to about 10 wt. %, wherein the weight of the basic amino acid is calculated based on its free base form.

11. The composition of claim 1 wherein the protease is a non-specific protease.

12. The composition of claim 1 wherein the protease is a specific protease.

13. The composition of claim 1 wherein the protease is trypsin or papain.

14. The composition of claim 1 wherein the protease inhibitor is serpin.

15. The composition of claim 3 wherein the fluoride ion source is selected from stannous fluoride, sodium fluoride, potassium fluoride, sodium monofluorophosphate, sodium fluorosilicate, ammonium fluorosilicate, amine fluoride, ammonium fluoride, titanium fluoride, hexafluorosulfate, and combinations thereof.

16. The composition of claim 3 wherein the fluoride ion source is present in an amount of about 0.01 wt, % to about 2 wt. % of the total composition weight.

17. The composition of claim 1 wherein the pH is about 6 to about 9.

18. The composition of claim 1 wherein the pH is approximately neutral.

19. The composition of claim 1 further comprising an abrasive or particulate.

20. The composition of claim 19 wherein the abrasive or particulate is selected from sodium bicarbonate, calcium phosphate, calcium sulfate, precipitated calcium carbonate, silica, iron oxide, aluminum oxide, perlite, plastic particles, polyethylene, and combinations thereof.

21. The composition of claim 19 wherein the abrasive is present in an amount of about 15 wt. % to about 70 wt, % of the total composition weight.

22. The composition of claim 1 further comprising an anionic surfactant selected from sodium lauryl sulfate, sodium ether lauryl sulfate, and mixtures thereof.

23. The composition of claim 22 wherein the anionic surfactant is present in an amount of from about 0.3% to about 4.5% by weight of the total composition weight.

24. The composition of claim 1 further comprising an additional surfactant selected from cationic, zwitterionic, and nonionic surfactants, and mixtures thereof.

25. The composition of claim 1 further comprising at least one humectant selected from glycerin, sorbitol and combinations thereof.

26. The composition of claim 1 further comprising at least one polymer.

27. The composition of claim 1 further comprising gum strips or fragments.

28. (canceled)

29. The composition of claim 1 further comprising an antibacterial agent selected from:
triclosan,
herbal extracts,
essential oils, rosemary extract, tea extract, magnolia extract, thymol, menthol, eucalyptol, geraniol, carvacrol, citral, hinokitol, catechol, methyl salicylate, epigallocatechin gallate, epigallocatechin, gallic acid, miswak extract, sea buckthorn extract and propolis,
bisguanide antiseptic, chlorhexidine, alexidine and octenidine,
quaternary ammonium compound, cetylpyridinium chloride (CPC), benzalkonium chloride, tetradecylpyridinium chloride (TPC) and N-tetradecyl-4-ethylpyridinium chloride (TDEPC),
phenolic antiseptics,
hexetidine,
octenidine,
sanguinarine,
povidone iodine,
delmopinol,
salifluor,
metal ion,
stannous salts,
copper salts and iron salts,
sanguinarine,
oxygenating agent,
buffered sodium peroxyborate or peroxycarbonate,
phthalic acid and its salts,
monoperthalic acid and its salts and esters,
ascorbyl stearate,
oleoyl sarcosine,
alkyl sulfate,
dioctyl sulfosuccinate,
salicylanilide,
domiphen bromide,
delmopinol,
octapinol and other piperidino derivatives,
nicin preparations,
chlorite salts;
and mixtures of any of the foregoing.

30. The composition of claim 29 wherein the antibacterial agent is present in an amount of about 0.01 to about 5 wt. % of the total composition weight.

31. The composition of claim 1 further comprising an anti-inflammatory compound, wherein the anti-inflammatory compound is an inhibitor of at least one of host pro-inflammatory factors selected from matrix metalloproteinases (MMP's), cyclooxygenases (COX), PGE2, interleukin 1 (IL-1), IL-1β converting enzyme (ICE), transforming growth factor β1 (TGF-β1), inducible nitric oxide synthase (iNOS), hyaluronidase, cathepsins, nuclear factor kappa B (NF-δB), and IL-1 Receptor Associated Kinase (IRAK).

32. The composition of claim 31 wherein the anti-inflammatory compound is selected from aspirin, ketorolac, flurbiprofen, ibuprofen, naproxen, indomethacin, aspirin, ketoprofen, piroxicam, meclofenamic acid, nordihydoguaiaretic acid, and mixtures thereof.

33. The composition of claim 1 further comprising an antioxidant, wherein the antioxidant is selected from the group consisting of Co-enzyme Q10, PQQ, Vitamin C, Vitamin E, Vitamin A, anethole-dithiothione, and mixtures thereof.

34. The composition of claim 1 further comprising a whitening agent selected from a whitening active selected from the group consisting of peroxides, metal chlorites, perborates, percarbonates, peroxyacids, hypochlorites, and combinations thereof.

35. The composition of claim 1 further comprising an agent that interferes with or prevents bacterial attachment, wherein the agent that interferes with or prevents bacterial attachment is selected from the group consisting of solbrol, chitosan and combinations thereof.

36. The composition of claim 3, wherein the calcium ion source is a soluble calcium salt selected from calcium sulfate, calcium chloride, calcium nitrate, calcium acetate, calcium lactate, and combinations thereof.

37. The composition of claim 3, wherein the potassium ion source is a physiologically acceptable potassium salt and is present in an amount effective to reduce dentinal sensitivity.

38. The composition of claim 1 wherein the composition is toothpaste.

39. The composition of claim 1 wherein the composition is a mouthwash.

40. The composition of claim 1 wherein the composition is a chewing gum.

41. A method to:
(i) reduce or inhibit formation of dental caries,
(ii) reduce, repair or inhibit pre-carious lesions of the enamel,
(iii) reduce or inhibit demineralization and promote remineralization of the teeth,
(iv) reduce hypersensitivity of the teeth,
(v) reduce or inhibit gingivitis,
(vi) promote healing of sores or cuts in the mouth,
(vii) reduce levels of acid producing bacteria,
(viii) increase relative levels of arginolytic bacteria,
(ix) inhibit microbial biofilm formation in the oral cavity,
(x) raise and/or maintain plaque pH at levels of at least pH 5.5 following sugar challenge,
(xi) reduce plaque accumulation,
(xii) treat, relieve or reduce dry mouth,
(xiii) clean the teeth and oral cavity
(xiv) reduce erosion,
(xv) whiten teeth,
(xvi) immunize the teeth against cariogenic bacteria; and/or
(xvii) promote systemic health, including cardiovascular health, comprising introducing into the oral cavity of a patient in need thereof the composition of claim 1.

42. The method of claim 41, wherein the protease hydrolyzes the peptide when introduced into the oral cavity.

43. The method of claim 41 wherein the protease is inactivated or diluted when the composition is introduced into the oral cavity.

44. The method of claim 41 wherein the composition is a toothpaste and is applied to the oral cavity with a tooth brush.

45. The composition of claim 3, wherein said potassium ion source is selected from potassium nitrate and potassium chloride.

46. The composition of claim 37, wherein said potassium ion source is selected from potassium nitrate and potassium chloride.
